(19) United States
(12) Patent Application Publication
(10) Pub. No.: US 2010/0151015 A1
(43) Pub. Date: Jun. 17, 2010

(54) Compositions Comprising Melperone and Controlled-Release Dosage Forms

(76) Inventors: Gopi M. Venkatesh, Vandalia, OH (US); Phillip J. Stevens, Englewood, OH (US); Jin-Wang Lai, Springborro, OH (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/639,470

(22) Filed: Dec. 16, 2009

Related U.S. Application Data
(60) Provisional application No. 61/122,830, filed on Dec. 16, 2008.

(51) Int. Cl. A61K 9/54; A61K 9/14; A61K 31/4515; A61P 25/18

(52) U.S. Cl. 424/458; 424/494; 424/497; 424/495; 514/317; 424/490; 424/493

(57) ABSTRACT

The present invention is directed to pharmaceutical compositions, and methods of making such compositions, comprising microparticles containing a core comprising melperone and a controlled-release coating. The present invention is also directed to pharmaceutical dosage forms comprising melperone, including orally disintegrating tablets, conventional tablets, and capsules, and methods for their preparation.

### CROSS REFERENCE TO RELATED APPLICATIONS

The present application claims priority to U.S. Provisional Application No. 61/122,830 filed Dec. 16, 2008, which is incorporated herein by reference in its entirety for all purposes.

### BACKGROUND OF THE INVENTION

Parkinson's disease (PD) is a progressive neurodegenerative disorder characterized by bradykinesia, rigidity, tremor, and abnormal posture and gait. The primary pathology of PD is the marked loss (80-90%) of dopaminergic neurons that provide dopaminergic innervation to the striatum, resulting in a potential decrease in dopamine activity and an imbalance in the dopamine/acetylcholine systems that control movement. Long-term treatments of PD movement disorders with dopamine precursors (anti-Parkinsonian agents), although successful, are thought to contribute to psychiatric side effects (psychosis). Psychotic symptoms characteristically observed in PD include hallucinations. Patients with PD who develop psychosis have a higher rate of morbidity and mortality (approaching 100%) and may cause considerable caregiver stress and/or may end up in nursing homes (see Barbato L et al., “Melperone in the treatment of iatrogenic psychosis in Parkinson's disease,” Funct. Neurol. (1996) 11:201-7 and Fernandez H. et al., “Treatment of psychosis in Parkinson's disease: safety considerations,” Drug Saf. (2003); 26(9):643-59, each of which is incorporated in their entirety by reference for all purposes).

Currently there are no compounds approved for the treatment of psychosis associated with PD. Although the use of quetiapine, an atypical antipsychotic agent, has shown efficacy in initial open-label studies, worsening of motor symptoms of PD was observed in up to 30% of patients. More options for treatment of psychosis in patients with PD are needed, particularly treatments that do not contribute to the worsening of motor symptoms in patients who already suffer from poor motor functioning.

In a proof-of-concept study, melperone has shown efficacy in the treatment of psychosis in patients with PD. The onset of efficacy was rapid (within a week), and efficacy with a mean daily dose of 37.5 mg was maintained over 2 years of treatment. Moreover, the motor symptoms were not worsened by melperone treatment. Its greater affinity for serotonin 5-HT₂ receptors relative to dopamine D2 receptors, minimal striatal activity, lack of significant clinical effect on plasma prolactin levels, low incidence of extrapyramidal side effect (EPS) or tardive dyskinesia make melperone a particularly attractive candidate to treat psychosis.

Melperone hydrochloride, 1-(4-fluorophenyl)-4-(4-methyl-1-piperidinyl)-1-butanone hydrochloride is a white crystalline powder having a wide spectrum of neuroleptic properties. Melperone hydrochloride has a pKa of 9.1 and a short plasma elimination half life of about 3 hrs upon a single oral dose administration. Its molecular weight is 299.82. Melperone is freely soluble in aqueous solutions in the pH range from about 1.2 up to about pH 6.8, but poorly soluble at higher pH values (i.e., above pH=7.5; see the table below for details).

Solubility (mg/mL) of Melperone HCl

| Media | Solubility (mg/mL) |
|---|---|
| pH 1.2 (0.1N HCl) | 700 |
| pH 4.5 (phosphate) | 800 |
| pH 6.8 (phosphate) | 800 |
| Purified Water | 800 |
| pH 7.0 (phosphate) | ≧100 |
| pH 7.1 (phosphate) | 51.3 |
| pH 7.5 (phosphate) | 25.6 |
| Ethanol | 88.5 |

Oral doses of melperone are rapidly absorbed (mean Tₘₐₓ: approximately 1.5-3.0 hours upon oral tablet administration). Within the 25-100 mg oral dose range, the pharmacokinetic profile is non-linear at higher doses, giving relatively higher Cₘₐₓ and AUC values than would be expected with linear pharmacokinetics. Due to extensive first-pass metabolism, the absolute oral bioavailability is about 50-70%. In healthy subjects, melperone metabolism is also rapid (t_{1/2}=3-4 hrs following single oral doses) and extensive, with only about 7% of the dose excreted unchanged in urine. The t_{1/2} is longer following chronic dosing; t_{1/2} at steady-state was approximately 7 hrs in patients with schizophrenia receiving 100 mg melperone three times daily (tid) and a variety of concomitant medications. Formulations of melperone permitting a once- or twice-daily dosage regimen are desirable in order to improve compliance in patients suffering from schizophrenia or in the treatment of psychosis associated with PD.

However, developing extended release dosage forms of melperone for once- or twice-daily administration is challenging for several reasons. Due to their extreme solubility, especially under acidic to neutral pH conditions, weakly basic drugs like melperone are rapidly released under acidic conditions in the stomach and often fail to sustain release long enough for once- or twice-daily dosing. Extending the release by providing thicker polymer coatings is problematic because such coatings can be difficult to manufacture, and/or increase the bulk of the dosage form (particularly for drugs such as melperone, which require the administration of relatively high doses).

### SUMMARY OF THE INVENTION

In one embodiment, the present invention relates to a pharmaceutical composition comprising one or more populations of controlled-release particles, wherein at least one population of controlled-release particles comprises a core comprising melperone or a pharmaceutically acceptable salt, ester, and/or solvate thereof, and a controlled-release coating disposed over the core, wherein the controlled-release coating comprises a water-insoluble polymer.

In another embodiment, the present invention relates to a method of preparing the pharmaceutical composition, comprising (a) preparing a core comprising melperone or a pharmaceutically acceptable salt, ester, and/or solvate thereof; and (b) coating the core with a controlled-release layer comprising a water-insoluble polymer.

In another embodiment, the present invention relates to a pharmaceutical dosage form comprising a pharmaceutical composition comprising one or more populations of controlled-release particles. Each particle in at least one population of controlled-release particles comprises a core comprising melperone (or a pharmaceutically acceptable salt, ester, and/or solvate thereof) and a controlled-release coating disposed over the core, wherein the controlled-release coating comprises a water-insoluble polymer, optionally in combination with a water-soluble polymer and/or an enteric polymer.

In another embodiment, the present invention relates to a pharmaceutical dosage form comprising at least two populations of drug particles. One population of drug particles comprises melperone-containing particles, while the second population comprises melperone-containing particles coated with a controlled-release layer. The controlled-release layer comprises a water-insoluble polymer, optionally in combination with a water-soluble polymer and/or an enteric polymer.

In another embodiment, the present invention relates to a pharmaceutical dosage form comprising at least one population of particles. Each particle of at least one population of particles comprises a core comprising melperone or a pharmaceutically acceptable salt, ester, and/or solvate thereof, coated with a controlled-release layer comprising a water-insoluble polymer.

In another embodiment, the present invention relates to a method of preparing the pharmaceutical dosage form. In a particular embodiment, the pharmaceutical dosage form is prepared by (a) mixing the melperone-containing particles described herein with rapidly dispersing granules comprising a saccharide and/or a sugar alcohol in combination with a disintegrant to form a compressible blend; and (b) compressing the blend into a tablet. In another embodiment, the pharmaceutical dosage form is prepared by filling the melperone-containing particles described herein into a hard-gelatin capsule.

### BRIEF DESCRIPTION OF THE DRAWINGS

FIG. 1 illustrates a cross-section of an embodiment of an SR/TPR bead comprising an inert core, a drug layer, a sealant layer, and a SR and TPR coating prepared in accordance with certain embodiments of the invention. The SR/TPR bead 10 comprises a lag-time coating 12 disposed over a sustained-release (SR) coating 14 disposed over a sealant layer 16, which is disposed over a drug layer comprising melperone HCl 18, coated on an inert core 20.

FIG. 2 is a schematic of a two-compartment pharmacokinetic model.

FIG. 3 shows the deconvoluted in vitro drug release profiles for melperone hydrochloride from a 2-compartment model of Example 1.

FIG. 4 shows the in vitro drug release profiles of melperone hydrochloride from TPR beads of Example 2.D (Lot# 1272-161) at two different controlled-release coating levels and Example 2.F (Lot# 1272-141) at two different controlled-release coating levels.

FIG. 5 illustrates the release profiles of melperone hydrochloride from SR and TPR beads of Example 3 (Lot nos. 1272-109, 1272-074, 1272-111, and 1272-121) at various controlled-release coating levels and various ratios of EC-10/PEG 400 or EC-10/HP-55/TEC (indicated by the parenthesis in the legend).

FIG. 6 illustrates the release profiles of melperone hydrochloride from the controlled release capsules of Example 4 (Lot nos. 1295-030, 1295-031, and 1295-032) and of Example 6 (PF414EA002).

FIG. 7 illustrates the drug release profiles of the ODTs of Example 5 (lot nos. 1295-055 and 1295-161) and Example 6 (PF417EA0001) (all prepared from 45-60 mesh sugar spheres), as well as the SR beads of Example 5.B (Lot# 1295-001).

### DETAILED DESCRIPTION OF THE INVENTION

All documents cited are incorporated herein by reference in their entirety for all purposes. The citation of any document is not to be construed as an admission that it is prior art with respect to the present invention.

The term “drug”, “active” or “active pharmaceutical ingredient” as used herein includes a pharmaceutically acceptable and therapeutically effective compound, pharmaceutically acceptable salts, stereoisomers and mixtures of stereoisomers, solvates (including hydrates), polymorphs, and/or esters thereof. When referring to a drug in the descriptions of the various embodiments of the invention, the reference encompasses the base drug, pharmaceutically acceptable salts, stereoisomers and mixtures of stereoisomers, solvates (including hydrates), polymorphs, and/or esters thereof.

The terms “orally disintegrating tablet” or “ODT” refers to a tablet which disintegrates rapidly in the oral cavity of a patient after administration, without the need for chewing. The rate of disintegration can vary, but is faster than the rate of disintegration of conventional solid dosage forms (e.g., tablets or capsules) which are intended to be swallowed immediately after administration, or chewable solid dosage forms.

The term “about”, as used herein to refer to a numerical quantity, includes “exactly”. For example, “about 60 seconds” includes 60 seconds, exactly, as well as values close to 60 seconds (e.g., 50 seconds, 55 seconds, 59 seconds, 61 seconds, 65 seconds, 70 seconds, etc.).

As used herein, the term “controlled-release” coating encompasses coatings that delay release, sustain release, prevent release, and/or otherwise prolong the release of a drug from a particle coated with a controlled-release coating. The term “controlled-release” encompasses “sustained-release” and “timed, pulsatile release.” As used herein, the term “controlled-release coating” encompasses a timed, pulsatile release or “lag-time” coating.

As used herein, the term “immediate-release core” refers to a core containing drug, optionally layered with a sealant layer, but not coated with a controlled-release coating. An “immediate-release core” can include drug crystals (or amorphous particles), granules of the drug with one or more excipients, or an inert core (e.g., a sugar sphere) layered with a drug (and an optional binder), a protective sealant coating, and an optional alkaline buffer layer. “Immediate release cores” have immediate release properties as described herein. Extended release particles (e.g., SR particles, TPR particles, etc.) can be prepared by coating immediate-release cores with an extended release coating.

As used herein, the term “immediate release” or IR refers to release of melperone greater than or equal to about 50% (especially if taste-masked for incorporation into an orally disintegrating tablet dosage form), preferably greater than about 75%, more preferably greater than about 90%, and in accordance with certain embodiments greater than about 95% of the active within about 2 hours, more particularly within about one hour following administration of the dosage form. In particular embodiments, “immediate release” particles released at least about 85% of the melperone contained within the immediate release particle within about 45 minutes when tested for dissolution in a USP Apparatus 1 (baskets at 100 rpm) or Apparatus 2 (paddles at 50 rpm) in 900 mL of 0.1 N HCl at 37° C. In other embodiments, the “immediate release” particles released at least about 85% of the melperone within about 15 minutes, when dissolution tested as described above.

The term “substantially masks the taste” in reference to the taste-masking layer of the IR particles (when present) refers to the ability of the taste masking layer to substantially prevent release of a bitter tasting drug in the oral cavity of a patient. A taste-masking layer which “substantially masks” the taste of the drug typically releases less than about 10% of the drug in the oral cavity of the patient, in other embodiments, less than about 5%, less than about 1%, less than about 0.5%, less than about 0.1%, less than about 0.05%, less than about 0.03%, less than about 0.01% of the drug. The taste-masking properties of the taste-masking layer of the compositions of the present invention can be measured in vivo (e.g., using conventional organoleptic testing methods known in the art) or in vitro (e.g., using dissolution tests as described herein). The skilled artisan will recognize that the amount of drug release associated with a taste-masking layer that “substantially masks” the taste of a drug is not limited to the ranges expressly disclosed herein, and can vary depending on other factors such as the perceived the bitterness of the drug and, e.g. the presence of flavoring agents in the composition.

The term “TPR particle” or “TPR bead” refers to a drug-containing particle, e.g., a drug-layered bead, drug-containing granulate, or drug particle, coated with a TPR (“timed pulsatile release”) coating. The TPR coating provides an immediate release pulse of the drug, or a sustained drug-release profile after a pre-determined lag time. The term “lag-time” refers to a time period immediately after administration of the drug-containing particle wherein less than about 10%, more particularly substantially none, of the drug is released from a particle. In some embodiments, a lag-time of from at least about 2 to 10 hours is achieved by coating the particle with, e.g. a combination of at least one water-insoluble polymer and at least one enteric polymer (e.g., a combination of ethylcellulose and hypromellose phthalate). The TPR layer can optionally contain a plasticizer.

The term “sustained-release coating” or “SR coating” refers to a coating providing sustained-release properties, e.g. a coating which slows the release of the drug from the drug-containing particle but does not provide an appreciable “lag-time.”

The term “substantially disintegrates” means a level of disintegration amounting to disintegration of at least about 50%, at least about 60%, at least about 70%, at least about 80%, at least about 90%, or about 100% disintegration of the ODT composition.

The term “rapidly dispersing tablet” as disclosed in this application refers to a conventional tablet that is orally administered either by swallowing or dispersing in a small amount of water. “Rapidly dispersing tablets” comprise at least one drug, and optionally a disintegrant, one or more fillers/diluents (e.g. including microcrystalline cellulose or mannitol). “Rapidly dispersing tablets” are distinguished from ODTs in that ODTs contain the rapidly dispersing microgranules described herein (comprising a saccharide and/or a sugar alcohol in combination with a disintegrant), whereas “rapidly dispersing tablets” do not (even though they may contain disintegrants and sugar alcohols). Because ODTs contain rapidly dispersing microgranules, ODTs disintegrate rapidly in the oral cavity upon exposure to saliva.

The terms “plasma concentration-time profile”, “Cₘₐₓ”, “AUC”, “Tₘₐₓ”, and “elimination half life” have their generally accepted meanings as defined in the FDA Guidance to Industry: Bioequivalence.

Unless indicated otherwise, all percentages and ratios are calculated by weight based on the total composition.

The term “disposed over” means that a second material is deposited over a first material, wherein the second material may or may not be in physical contact with the first material. Thus it is possible, but not necessary, that an intervening material lies between the first and second materials. The term “disposed on” means that a second material is deposited directly onto a first material, wherein the first and second materials are in physical contact and no intervening material lies between them. The term “disposed between” means that an intermediate material lies between two other materials but is not necessarily in physical contact with them. Thus it is possible, but not necessary, that an intervening material lies between the intermediate material and either or both of the two materials surrounding it.

In one embodiment, the present invention relates to a pharmaceutical composition comprising a plurality of controlled-release particles, wherein each particle in at least one population of particles comprises a core comprising melperone or a pharmaceutically acceptable salt, solvate, and/or ester thereof, and a controlled-release coating disposed over the core. In particular embodiments, the controlled-release coating comprises a water-insoluble polymer. In other embodiments, the controlled-release coating comprises a water-insoluble polymer in combination with a water-soluble polymer. In still other embodiments, the controlled-release coating comprises a water-insoluble polymer in combination with an enteric polymer.

Within the controlled-release particle, a sealant layer can be disposed between the controlled-release coating and the melperone layer, or can be disposed on or between one or more layers disposed between the controlled release coating and the melperone layer. In a particular embodiment, the controlled-release coating is disposed on a sealant layer, which in turn is disposed on the melperone-containing core. In one embodiment, the sealant layer comprises a hydrophilic polymer.

Non-limiting examples of suitable hydrophilic polymers include hydroxypropylcellulose (e.g., Klucel® LF), hydroxypropyl methylcellulose or hypromellose (e.g., Opadry® Clear or Pharmacoat™ 603), vinylpyrrolidone-vinylacetate copolymer (e.g., Kollidon® VA 64 from BASF), and ethylcellulose, e.g. low-viscosity ethylcellulose. The sealant layer can constitute from about 1% to about 10% of the weight of the melperone-containing, sealant-coated core, for example about 1%, about 2%, about 3%, about 4%, about 5%, about 7%, about 8%, about 8%, or about 10%, inclusive of all ranges and subranges therebetween.

The melperone-containing particles of the present invention comprise a controlled-release coating comprising a water-insoluble polymer. In another embodiment, the controlled-release coating comprises the water-insoluble polymer in combination with a water-soluble and/or an enteric polymer. In the embodiments, the controlled-release coating sustains release of melperone over from about 8 hours to about 20 hours, depending on the average particle size of the inert cores, the composition of the controlled-release coating, and the level of controlled-release coating (when tested using a two-stage dissolution method; 700 mL of 0.1N HCl for the first 2 hours and thereafter in 900 mL of aqueous pH 6.8 solution obtained by adding 200 mL of a pH modifier to the 0.1N HCl solution). This release profile is suitable for a once- or twice-daily dosing regimen.

Non-limiting examples of suitable water-insoluble polymers include ethylcellulose, cellulose acetate, cellulose acetate butyrate, polyvinyl acetate, neutral methacrylic acid-methylmethacrylate copolymers, and mixtures thereof. In one embodiment, the water-insoluble polymer comprises ethylcellulose. In another embodiment, the water-insoluble polymer comprises ethylcellulose with a mean viscosity of 10 cps in a 5% solution in 80/20 toluene/alcohol measured at 25° C. on an Ubbelohde viscometer. The water-insoluble polymer of the sustained-release coating provides a weight gain from about 3% to about 40%, including about 3%, about 5%, about 7%, about 10%, about 12%, about 15%, about 17%, about 20%, about 22%, about 25%, about 27%, about 30%, about 35%, and about 40%, inclusive of all ranges and subranges therebetween. In one example, the sustained-release microparticle may have a sustained-release coating of a plasticized water-insoluble polymer, such as ethylcellulose (EC-10), at about 5-50% by weight to sustain the release of melperone over about 4-20 hours.

In one embodiment, the water-insoluble polymer of the controlled-release coating further comprises a plasticizer. Non-limiting examples of suitable plasticizers include triacetin, tributyl citrate, triethyl citrate, acetyl tri-n-butyl citrate, diethyl phthalate, polyethylene glycol, castor oil, dibutyl sebacate, monoacetylated and diacetylated glycerides (e.g., Myvacet® 9-45), and mixtures thereof. When used in an embodiment of the present invention, the plasticizer may constitute from about 3% to about 30% by weight of the water-insoluble polymer. In another embodiment, the plasticizer constitutes from 10% to about 25% by weight of the water-insoluble polymer. In still other embodiments, the amount of plasticizer relative to the weight of the water-insoluble polymer is about 3%, about 5%, about 7%, about 10%, about 12%, about 15%, about 17%, about 20%, about 22%, about 25%, about 27%, and about 30%, inclusive of all ranges and subranges therebetween. One of ordinary skill in the art would know to select the type of plasticizer based on the polymer or polymers and nature of the coating system (e.g., aqueous or solvent-based, solution or dispersion-based and the total solids).

In some embodiments, the controlled-release coating comprises a water-insoluble polymer in combination with a water-soluble polymer. In one embodiment, the ratio of the water-insoluble polymer to the water-soluble polymer ranges from about 95/5 to about 50/50, including the range of about 90/10 to about 60/40. In another embodiment, the water-insoluble and water-soluble polymers in combination constitute from about 3% to about 50% by weight of the coated bead, including the ranges from about 10% to about 50%, about 3% to about 30%, and from about 5% to about 30%. In other embodiments, the amount of water-insoluble and water-soluble polymers in combination constitute about 3%, about 5%, about 7%, about 10%, about 12%, about 15%, about 17%, about 20%, about 22%, about 25%, about 27%, about 30%, about 35%, about 40%, about 45%, and about 50% of the weight of the coated core, inclusive of all ranges and subranges therebetween.

Non-limiting examples of suitable water-soluble polymers include polyvinylpyrrolidone (e.g., Povidone K-25), polyethylene glycol (e.g., PEG 400), hydroxypropyl methylcellulose, and hydroxypropylcellulose. In one embodiment, the sustained-release coating provides a drug release sustained over from about 8 to about 20 hours when tested using a two-stage dissolution method (700 mL of 0.1N HCl for the first 2 hours and thereafter in 900 mL of aqueous pH 6.8 solution obtained by adding 200 mL of a pH modifier to the 0.1N HCl solution), suitable for a once- or twice-daily dosing regimen.

In another embodiment, the controlled-release coating comprises a water-insoluble polymer in combination with a gastrosoluble pore-former and sustains release of the melperone. An example of a gastrosoluble pore-former is calcium carbonate. Other suitable gastrosoluble pore-formers include sodium chloride, calcium phosphate, calcium saccharide, calcium succinate, calcium tartrate, ferric acetate, ferric hydroxide, ferric phosphate, magnesium carbonate, magnesium citrate, magnesium hydroxide, magnesium phosphate, etc.

In one embodiment, the pharmaceutical composition comprises a plurality of controlled-release particles, wherein at least a portion of the controlled-release particles comprise: (a) a core comprising melperone hydrochloride; and (b) a controlled-release coating disposed over the melperone hydrochloride-containing core, comprising ethylcellulose.

In another embodiment, the controlled-release coating comprises a water-insoluble polymer in combination with an enteric polymer, thereby providing a delayed or a timed, pulsatile release (TPR) of melperone. This type of controlled-release coating (i.e., the combination of water-insoluble and enteric polymers) is an embodiment of a “lag-time” coating, and particles or microparticles coated with the lag-time coating may be referred to herein as TPR particles or microparticles. The term “lag-time” refers to a time period wherein less than about 10% of the melperone is released from the particle or microparticle after ingestion of the dosage form or after exposure to simulated body fluid(s). In one embodiment, the lag-time coating is deposited directly onto the melperone-containing particle. In another embodiment, the lag-time coating is deposited directly onto one or more layers (e.g., a sealant layer) coated onto the melperone-containing particle. In some embodiments, the ratio of the water-insoluble polymer to enteric polymer ranges from about 10:1 to about 1:4, including the ranges of from about 9:1 to about 1:3 and from about 3:1 to about 1:1. In other embodiments, the water-insoluble and enteric polymers in combination constitute from about 5% to about 60% by weight of the coated core, including the ranges of from about 10% to about 60%, and from about 10% to about 50%.

Non-limiting examples of suitable enteric polymers include cellulose acetate phthalate, hydroxypropyl methylcellulose phthalate, hydroxypropyl methylcellulose acetate succinate, polyvinyl acetate phthalate, pH-sensitive methacrylic acid-methylmethacrylate copolymers, shellac, and mixtures thereof. (The term “pH sensitive” refers to polymers which exhibit pH dependent solubility.) These enteric polymers may be used as a dry powder or an aqueous dispersion. Some commercially available materials that may be used are methacrylic acid copolymers sold under the trademark Eudragit (L100, 5100, L30D) manufactured by Rohm Pharma, Cellacefate (cellulose acetate phthalate) from Eastman Chemical Co., Aquateric (cellulose acetate phthalate aqueous dispersion) from FMC Corp., and Aqoat (hydroxypropyl methylcellulose acetate succinate aqueous dispersion) from Shin Etsu K.K. In one embodiment, the TPR-coating comprises ethylcellulose (e.g., EC-10) as the water-insoluble polymer and hypromellose phthalate (e.g., HP-55) as the enteric polymer.

In one embodiment, the TPR microparticles provide a lag time of from about 1 hour to about 10 hours, including from about 2 hours to about 7 hours, from about 2 hours to about 4 hours (“shorter lag time”), and from about 7 hours to about 8 hours (“longer lag time”). In another embodiment, the TPR microparticles release melperone over a period of about 4 hours to about 16 hours in the gastrointestinal tract after a lag time of about 1 hour to about 10 hours following oral administration.

In another embodiment, the microparticles contain an outer, lag-time coating disposed on a sustained-release coating. Microparticles according to this embodiment begin to release melperone in the higher pH of the intestine, followed by sustained-release of melperone.

The melperone release profiles of SR and TPR microparticles may be determined by dissolution testing in a USP Apparatus 1 or 2 using a two-stage dissolution medium (first 2 hours in 700 mL of 0.1N HCl at 37° C. followed by dissolution testing at pH 6.8 obtained by the addition of 200 mL of a pH modifier). Melperone release over time can be determined using various methods; for example, by HPLC on samples pulled at selected time points.

The SR or TPR coating contributes to the control of melperone dissolution at the drug interface and hence melperone release from the microparticles. The achievable lag time or sustained-release time depends on the composition and thickness of the sustained-release coating, and/or the composition and thickness of the lag-time coating. Specific factors that can affect achieving optimal once-daily dosage forms include, but are not limited to, the melperone's pKa and its solubility.

In another embodiment, the microparticles contain a compressible coating disposed on the controlled-release coating (or disposed on the outer-most coating, if the controlled-release coating is further coated with a TPR coating). The compressible coating comprises a polymer, for example selected from the group consisting of hydroxypropylcellulose, poly(vinyl acetate-vinyl pyrrolidone), polyvinyl acetate, and plasticized low-viscosity ethylcellulose latex dispersion. This coating may be applied, for example, by fluid-bed coating with a plasticized aqueous dispersion of ethylcellulose. The function of the compressible coating is to maintain membrane integrity during compression with rapidly dispersing microgranules. The amount of compressible coating, when present, can be about 1%, about 2%, about 3%, about 4%, about 5%, about 6%, about 7%, about 8%, about 9%, or about 10%, inclusive of all values, ranges and subranges therebetween.

The microparticle core comprises melperone. In some embodiments, the core can take the form of an inert bead, a microgranule, or a melperone crystal. In one embodiment, the core comprises an inert bead, coated with a layer comprising melperone or a pharmaceutically acceptable salt, solvate, and/or ester thereof. The inert bead can comprise sugar, lactose, microcrystalline cellulose, mannitol-microcrystalline cellulose, silicon dioxide, etc. The core can have any suitable size. In some embodiments, the core has an average particle size of not more than 400 μm, or, in other embodiments, not more than 350 μm. In other embodiments, the microparticle core comprises an inert bead coated with a melperone layer, wherein the melperone layer further comprises a polymeric binder. Typically, the binder is used at a concentration of about 0.5 to 10% by weight. The drug concentration may vary depending on the application but typically will be used at concentrations from about 5 to 30% by weight depending on the viscosity of the coating formulation. The polymeric binder can be selected from the group consisting of hydroxypropylcellulose, povidone, methylcellulose, hydroxypropyl methylcellulose, carboxyalkylcellulose, polyethylene oxide, starch (e.g., corn starch or gelatinized corn starch), and a polysaccharide. The ratio of melperone to the polymeric binder can range from about 85:15 to about 100:0 (no binder).

The individual SR or TPR polymeric coatings on IR beads will vary from about 5 to 50% by weight depending on the solubility of the active, particle size of inert cores, drug load, composition of the barrier coat, and required lag-time. In one embodiment, the IR beads may be provided with a barrier-coat of a plasticized water-insoluble polymer, such as ethylcellulose (EC-10), at about 5-50% by weight to sustain the drug release over about 5-20 hours. In certain other embodiments, the IR beads may be provided with a barrier-coat of a plasticized ethylcellulose and hydroxypropyl methylcellulose (hypromellose) phthalate (HP-55) at about 10-50% by weight or the IR beads are coated with ethylcellulose (EC-10) at 5-20% w/w and subsequently provided with an outer lag-time coating of EC-10/HP-55/plasticizer at about 45.5/40/14.5 for a weight gain of about 30-50% by weight to controls the drug-release following the lag-time. The composition of the membrane layer and the individual weights of the polymers are important factors to be considered for achieving a desired drug-release profile and lag time prior to appreciable drug release.

The pharmaceutical compositions described herein can further comprise rapidly disintegrating granules comprising a saccharide and/or a sugar alcohol in combination with a disintegrant. Suitable disintegrants include, for example, disintegrants selected from the group consisting of crospovidone, sodium starch glycolate, crosslinked sodium carboxymethylcellulose, low-substituted hydroxypropylcellulose, and combinations thereof. Suitable saccharides and/or sugar alcohols may be selected from the group consisting of lactose, sucralose, sucrose, maltose, mannitol, sorbitol, xylitol, maltitol, and combinations thereof. The ratio of the disintegrant to the saccharide and/or sugar alcohol in the rapidly dispersing microgranules ranges from about 1/99 to about 10/90, and in some embodiments is about 5/95 (by weight). In some embodiments, the disintegrant or the saccharide and/or sugar alcohol, or both, can be present in the form of microparticles having an average particle size of about 30 μM or less. The ratio of the melperone-containing microparticles to the rapidly disintegrating granules can range from about 1:6 to about 1:2.

In accordance with particular aspects of the present invention, the pharmaceutical multiparticulate dosage form may comprise only the SR beads. In certain embodiments of the present invention, the pharmaceutical multiparticulate dosage form may include an IR bead population, a first TPR bead population, and an SR bead population or a second TPR bead population. In certain embodiments, the ratio of IR bead population to the first TPR bead population to the SR bead or second TPR bead population may vary from about 10:90:0 to about 40:10:50. In still other embodiments, the pharmaceutical multi-particulate dosage form may include an IR bead population and an SR bead population. In other embodiments, the pharmaceutical multi-particulate dosage form may include an IR bead population and a TPR bead population.

In another embodiment, the present invention relates to pharmaceutical dosage forms comprising the microparticles described herein. The pharmaceutical dosage forms include orally disintegrating tablets (ODTs), conventional tablets, and capsules. When the pharmaceutical dosage form is a conventional tablet, the conventional tablet comprises microparticles of the present invention, combined as needed with any pharmaceutically acceptable excipient(s), such as fillers, diluents, lubricants, compression aids, etc. when the pharmaceutical dosage form is a capsule, a capsule is filled with at least one population of microparticles of the present invention, combined as needed with any pharmaceutically acceptable excipients. The capsule can be a gelatin capsule, an HPMC capsule, etc.

When the pharmaceutical dosage form takes the form of an ODT, the ODT substantially disintegrates within about 60 seconds after contact with saliva in the oral cavity or with simulated saliva fluid. In another embodiment, the ODT substantially disintegrates within about 30 seconds. Disintegration is tested according to USP <701> Disintegration Test. In one embodiment, the ODT comprises a therapeutically effective amount of melperone, wherein after administration the ODT substantially disintegrates in the oral cavity of a patient forming a smooth, easy-to-swallow suspension having no gritty mouthfeel or aftertaste and provides a target PK profile (i.e., plasma concentration vs. time plot) of melperone suitable for a once- or twice-daily dosing regimen.

When the pharmaceutical dosage form is a tablet, it preferably has a friability of less than about 1%. When the dosage form is an ODT, the ODT may also include pharmaceutically acceptable excipients typically used in disintegrating tablet formulations such as compressible diluents, fillers, coloring agents, and optionally a lubricant.

An ODT can comprise one or more populations of SR or TPR microparticles described herein, or mixtures thereof, combined with rapidly disintegrating microparticles. The ODTs may further comprise one or more populations of IR particles, in addition to the SR or TPR microparticles. For example, the pharmaceutical dosage form may comprise: SR microparticles in combination with rapidly disintegrating granules; TPR microparticles in combination with rapidly disintegrating granules; IR microparticles, SR microparticles, and rapidly dispersing granules; IR microparticles, TPR microparticles, and rapidly dispersing granules; or IR microparticles, SR microparticles, and one or more populations of TPR microparticles which may have the same or different lag times (e.g., short lag-time TPR microparticles and long lag-time TPR microparticles), combined with rapidly dispersing granules. These different combinations of microparticles can achieve different desired melperone release profiles. For example, a once-daily dosage form of melperone with an elimination half-life of about 7 hours may contain a mixture of an IR bead population which provides an immediate-release pulse, a second SR bead or TPR bead population with a shorter lag time (about 2-4 hours), which provides a rapid sustained-release profile, and a third TPR bead population with a longer lag time (about 7-8 hours), which allows typically a delayed, sustained-release profile over about 8-12 hours, to maintain acceptable plasma concentrations at 12-24 hours.

Other dosage forms such as capsules or conventional tablets can also include combinations of IR and SR and/or TPR particles. When IR particles are present in the pharmaceutical dosage form (e.g., capsules, conventional tablets, or ODTs), the ratio of IR particles to SR and/or TPR particles ranges from about 0:100 (no IR particles) to about 50:50.

When the ODT contains IR particles, the IR particles may be taste-masked by applying a taste-masking layer that substantially masks the taste of melperone contained in the particle. These taste-masked IR particles release not more than about 10% of the melperone contained in the IR particles in 3 minutes (the longest typical residence time anticipated for the ODT in the buccal cavity) when dissolution tested in simulated saliva fluid (pH ˜6.8) while releasing not less than about 75% of the melperone contained in the IR particles in about 60 minutes when dissolution tested in 0.1N HCl.

The taste-masking layer comprises a water-insoluble polymer (e.g., ethylcellulose) which prevents release of the melperone in the oral cavity, but does not substantially hinder release in the gastrointestinal tract. If present, the coating of water-insoluble polymer on the IR particles may further comprise a plasticizer. It can further comprise a gastrosoluble pore-former (e.g., calcium carbonate), for example in accordance with the disclosure in the co-pending U.S. patent application Ser. No. 11/213,266 filed Aug. 26, 2005 (Publication No. U.S. 2006/0105038 published May 18, 2006) or by fluid-bed coating with a water-insoluble polymer (e.g., ethylcellulose with a mean viscosity of 10 cps) alone or in combination with a gastrosoluble polymer (e.g., Eudragit E100 or EPO), for example in accordance with the disclosure in the co-pending U.S. patent application Ser. No. 11/248,596 filed Oct. 12, 2005 (Publication No. U.S. 2006/0078614 published Apr. 13, 2006) or a gastrosoluble pore-former (e.g., calcium carbonate), for example in accordance with the disclosure in the co-pending U.S. patent application Ser. No. 11/256,653 filed Oct. 21, 2005 (Publication No. U.S. 2006/0105039 published May 18, 2006). Each of these applications set forth herein are incorporated by reference in their entirety for all purposes.

The ODTs described herein can have one or more of the following properties or compositions: (i) disintegrating on contact with saliva in the oral cavity in about 60 seconds, forming a smooth, easy-to-swallow suspension comprising taste-masked melperone-containing particles; (ii) disintegrating within about 30 seconds when tested by the USP <701> Disintegration Test; (iii) taste-masked IR particles, if present, provide rapid, substantially complete release of the melperone dose upon entry into the stomach (e.g., typically greater than about 75% in about 60 minutes); and/or (iv) a melperone-containing SR and/or TPR particles which sustain and/or delay release of melperone in the gastrointestinal tract.

In another embodiment, the present invention is directed to methods of preparing a pharmaceutical composition of the microparticles described herein. In one embodiment, the method comprises: (a) preparing a core comprising melperone; and (b) coating the core of with a controlled-release layer. The step of preparing the core may be accomplished by any of the methods known in the art; for example, layering an inter bead (e.g., sugar, lactose, microcrystalline cellulose, mannitol-microcrystalline cellulose, lactose-microcrystalline cellulose, silicon dioxide, etc.) with a solution comprising the drug and optionally a polymeric binder (e.g., by fluid-bed or pan coating); granulating the drug with an appropriate diluent (e.g., microcrystalline cellulose); extruding and spheronizing the drug mixture; compressing the drug into mini-tablets of about 1-2 mm in diameter; or simply obtaining drug crystals of the desired particle size (e.g., about 50-500 μm, including about 100-400 μm). Non-limiting examples of suitable inert particles used to prepare the active core include sugar spheres, lactose spheres, cellulose spheres, mannitol-MCC (microcrystalline cellulose) spheres and silicon dioxide spheres with a suitable particle size distribution (for example 20-25 mesh sugar spheres for making coated beads for incorporation into a capsule formulation and 60-80 mesh sugar spheres or 100-200 μm cellulosic spheres for making coated beads for incorporation into an ODT formulation). In most embodiments, when the core comprises an inert bead, the particle size is about 20-150 mesh, for example about 20 mesh, about 25 mesh, about 30 mesh, about 35 mesh, about 40 mesh, about 45 mesh, about 50 mesh, about 55 mesh, about 60 mesh, about 65 mesh, about 70 mesh, about 75 mesh, about 80 mesh, about 85 mesh, about 90 mesh, about 95 mesh, about a hundred mesh, about 110 mesh, about 120 mesh, about 130 mesh, about 140 mesh, or about 150 mesh, inclusive of all values, ranges, and subranges therebetween.

In another embodiment, the method of preparing of preparing a pharmaceutical composition of microparticles further comprises coating the melperone-containing core with a sealant layer before coating with a controlled-release layer.

In another embodiment, the method of preparing a pharmaceutical composition of microparticles further comprises applying a compressible coating comprising at least one hydrophilic polymer, wherein the compressible coating is disposed over the controlled-release layer.

In one embodiment, the method is used to prepare a microparticle with a sustained-release coating. In this embodiment, the controlled-release coating of step (b) comprises a water-insoluble polymer and optionally a water-soluble polymer for a weight gain of from about 3% to about 40% to give a SR microparticle. In other embodiments, the coating weight (weight gain) of the controlled-release coating can be about 3%, about 5%, about 10%, about 15%, about 20%, about 25%, about 30%, about 35%, about 40%, inclusive of all values, ranges, and subranges therebetween.

In another embodiment, the method is used to prepare microparticles with a timed, pulsatile release (TPR) coating, or lag-time coating. In this embodiment, the controlled-release coating of step (b) comprises a water-insoluble polymer and an enteric polymer for a weight gain of from about 10% to about 60% to give a TPR microparticle. In another embodiment, the method is used to prepare microparticles with a sustained-release coating underlying an outer timed-pulsatile release coating. In this embodiment, the controlled-release coating of step (b) comprises a water-insoluble polymer and optionally a water-soluble polymer for a weight gain of from about 3% to about 30% (or about 3%, about 5%, about 10%, about 15%, about 20%, about 25%, about 30%, inclusive of all values, ranges, and subranges therebetween) to give a sustained-release microparticle. This sustained-release microparticle can optionally be coated with a layer comprising a water-insoluble polymer and an enteric polymer to give a SR/TPR microparticle.

In another embodiment, the present invention relates to a method of preparing a pharmaceutical dosage form comprising: (a) mixing the melperone-containing microparticles described herein with rapidly dispersing granules comprising a saccharide and/or sugar alcohol in combination with a disintegrant; and (b) compressing the resulting mixture into a tablet to provide an ODT. In still another embodiment, the pharmaceutical dosage form may be prepared by filling a capsule (e.g. a hard-gelatin capsule) with the microparticles described herein, or by compressing the melperone-containing microparticles described herein with pharmaceutically acceptable excipients to provide a conventional tablet.

### EXAMPLES

### Example 1

### Deconvoluted In Vitro Drug Release Profiles for Melperone QD

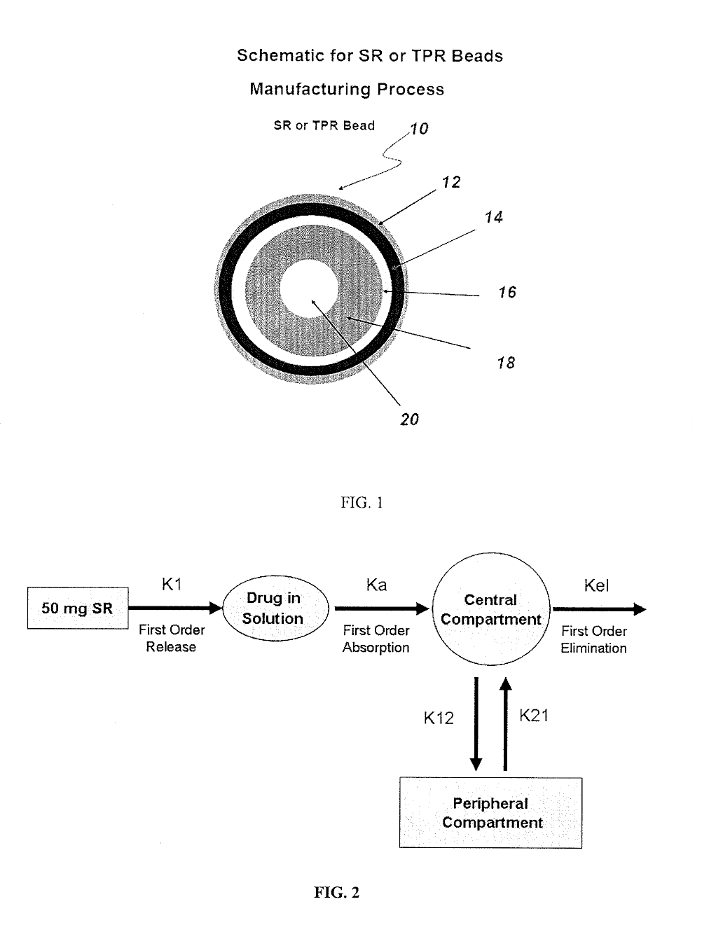

A pharmacokinetic evaluation was undertaken to identify a set of theoretical in vitro drug-release profiles that would be suitable for a once- or twice-daily dosage form of melperone. Using human plasma concentration-time data upon oral single dose administration or at steady state and/or upon an intravenous (IV) profile, melperone was found to fit a two-compartment model (see Table 1, below, and FIG. 2). Oral data (PO) shows the absence of a well-defined distribution phase, but both IV and PO data could be fit simultaneously to the two-compartment model shown below. Using WinNonlin Software, PK parameter estimates and predictions of both PO and IV data were performed as shown in Table 1. From these estimates, equations for simulated profiles were generated and are shown in FIG. 3. Rate constants 0.29, 0.19, and 0.14 per hour refer to fast CR, medium CR, and slow CR, respectively. Formulations with in vitro drug-release profiles that mimicked the simulated profiles of FIG. 3 were developed and tested in a PK study in adult healthy subjects.

TABLE 1
Parameter estimates of 20 mg IV and 50 mg Peroral Melperone

| | Estimate | StdError | CV % |
|---|---|---|---|
| Volume | 205406 | 4941.2 | 2.41 |
| Kₐ | 0.758 | 0.136 | 18.00 |
| K₁₀ | 0.935 | 0.053 | 5.69 |
| K₁₂ | 4.730 | 0.207 | 4.37 |
| K₂₁ | 1.358 | 0.073 | 5.35 |
| α | 6.838 | 0.277 | 4.05 |
| β | 0.186 | 0.014 | 7.57 |
| A | 80.205 | 2.188 | 2.73 |
| B | 17.164 | 0.598 | 3.49 |
| Tlag | 0.448 | 0.021 | 4.62 |

### Example 2

### 2.A Melperone IR Beads (60-80 mesh) at a Drug Load of 25%

Hydroxypropyl cellulose (Klucel LF, 42.9 g) was slowly added to ethanol (357.2 g while stirring vigorously for 10 minutes, and then purified water (100 g) was slowly added to the dissolved Klucel. Melperone HCl (357.2 g) was dissolved in purified water (3114.8 g), and then the binder solution was added to the melperone solution while stirring for additional 10 minutes. A Glatt GPCG 3 equipped with a 7″ bottom spray Wurster insert, 7 13/16″ partition column, ‘C’ air distribution plate covered with a 200 mesh product retention screen, and 16 mm tubing was charged with 1000 g of 60-80 mesh sugar spheres. The sugar spheres were layered with the melperone/Klucel solution while maintaining the product temperature at about 37-38° C. and inlet air volume 45-50 CFM. The flow rate was increased from 8 mL/min to 18 mL/min at an atomization air pressure of 1.25 bar and nozzle port of 1.0 mm. A protective sealant layer at 2% w/w of hydroxypropylcellulose Klucel® LF dissolved in 85/15 acetone/purified water at 7% solids was applied over the drug-layered beads. The beads were then dried in the unit for 10 min to drive off residual solvent/moisture and sieved through 30-80 mesh screens.

### 2.B Melperone SR Beads

The IR beads (1000 g) from 2.A were coated with a solution of EC-10 (Ethocel Standard Premium 10 cps, ethylcellulose of viscosity of approximately 10 cps from Dow Chemicals; 230 g) and PEG 400 (25.6 g) dissolved in acetone (2180.3 g)-water (384.7 g) mixture (6% solids) for a weight gain of 20% (relative to the weight of the coated bead), in a Glatt GPCG 3 equipped with a 6″ bottom spray Wurster insert as described above in 2.A, with an inlet temperature of 45-48° C. and inlet air volume at 40 CFM. Samples of SR coated beads were pulled at SR coating weight gains of 5%, 10%, and 15%. The SR coated beads were dried in the unit for about 5 minutes and sieved through 25±80 mesh screens.

### 2.C Melperone Hydrochloride IR Beads (60-80 mesh, Drug Load: 25% w/w)

Melperone HCl (1027.4 g) was slowly added to 50/50 acetone/water (3424.7 g each) while mixing to dissolve. 60-80 mesh sugar spheres (3000 g) were coated with the drug solution in a Glatt GPCG 5 equipped with 9″ bottom spray Wurster insert, 14″ partition column, 1″ partition gap, port diameter of 1 mm, air pressure of 1.4 bar, ‘D’ air distribution plate covered with a 200 mesh product retention screen and 16 mm tubing, while maintaining the product temperature at 32-34° C. and inlet air volume at 100 CFM. The spray rate was increased from about 10 mL/min to 30 mL/min. The drug-layered beads were provided with a protective seal-coat of hydroxypropylcellulose (Klucel LF, dissolved in 85/15 acetone/water at 7% solids) for a 2% weight gain to form IR beads (drug load: 25% w/w).

### 2.D Melperone Hydrochloride TPR Beads (Controlled-Release Coating: 35% by Weight)

The resulting melperone hydrochloride IR beads (from 2.C, above; 1000 g) were coated with a TPR coating solution prepared by first adding ethylcellulose (EC-10, 388.9 g) slowly to acetone (5000.4 g) to dissolve while stirring. HP-55 (111.1 g) was slowly added until dissolved. Water was then added (555.62 g), and finally TEC (55.6 g) was added with stirring for at least 10 min, to provide an acetone/water solution (10% solids) in a Glatt GPCG3 while maintaining the product temperature at 32-33° C., air volume at 35-40 CFM and a flow rate of 10 mL/min with a ramp-up to 20 mL/min. A sealant coating of Klucel LF was applied for a weight gain of about 2% w/w. The resulting TPR beads (Lot# 1272-161) were dried in the Glatt at the same temperature to drive off residual solvent and then sieved.

### 2.E Melperone Hydrochloride IR Beads (Drug Load: 25% by Weight)

Melperone hydrochloride (342.5 g) was slowly added to 50/50 acetone/water (1151.7 g each) to dissolve. 60-80 mesh sugar spheres (342.5 g) were coated with the drug solution in Glatt GPCG 3 equipped with a 7″ bottom spray Wurster insert as disclosed in 2.A above, and followed by a protective sealant coating of Klucel LF dissolved in 85/15 acetone/water (7% solids) at 2% by weight.

### 2.F Melperone Hydrochloride TPR Beads (Controlled-Release Coating: 35% by Weight)

Melperone hydrochloride IR beads (1000 g) were TPR coated by spraying a solution of 80/10/10 EC-10/HP-55/TEC (ethylcellulose (10 cps)/Hypromellose phthalate (HP-55)/Triethyl citrate) dissolved in 90/10 acetone/water (10% solids) for a weight gain of 35% (relative to the weight of the coated bead) in a Glatt GPCG 3 equipped with a 7″ bottom spray Wurster insert as described above in 2.B, with an inlet temperature of 40-44° C. and inlet air volume at 35 CFM. Samples were pulled at 22.5%, 25%, 27.5%, and 30% coating weights for drug release testing. Finally, a seal coat of Klucel LF was applied (2% w/w) to produce TPR beads of Lot# 1272-141.

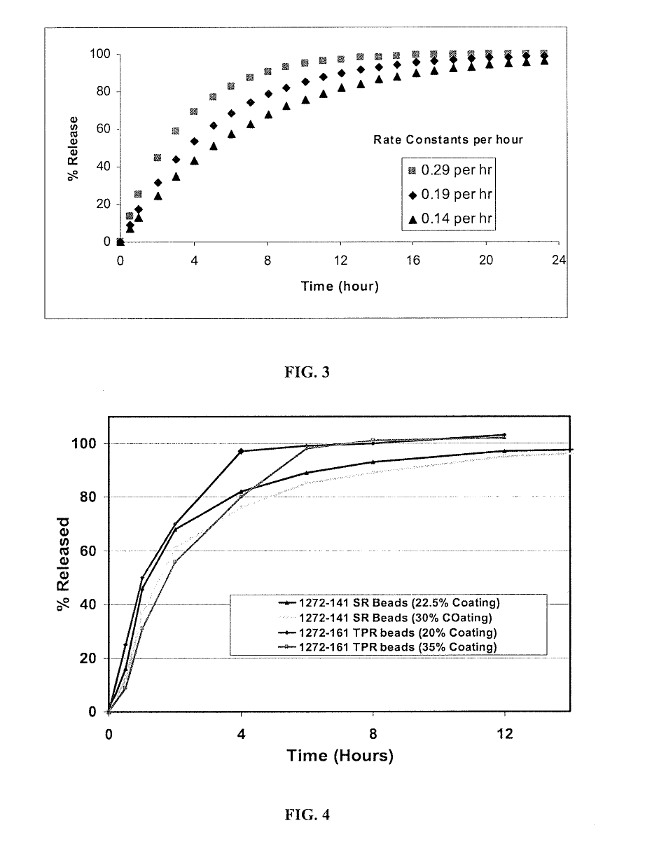

FIG. 4 shows the drug release profiles from the TPR beads (lot# 1272-141 at 22.5% and 30% coating and lot# 1272-161 at 20% and 35% coating) when dissolution tested using the 2-stage methodology disclosed above.

### Example 3

### 3.A Melperone HCl IR Beads (Drug Load: 25% by Weight)

Melperone HCl (1027.4 g) was slowly added to 50/50 acetone/water (3424.7 g each) while stirring until dissolved. A Glatt GPCG 5 equipped with a 9″ bottom spray Wurster insert, 25 mm partition gap, 14 mm tubing, bottom air distribution ‘D’ plate with 200 mesh screen, atomization air pressure of 1.30 bar and 1.0 mm nozzle diameter was charged with 3000 g of 25-30 mesh sugar spheres, which were sprayed with the melperone coating solution at 8 to 2037 mL/min while maintaining the product temperature at 34±2° C. and fluidization air volume at 100 CFM. A seal coat of Klucel LF dissolved in acetone/water was applied for a weight gain of 2%, and the resulting IR beads were dried to remove residual moisture and acetone.

### 3.B Melperone HCl SR Beads (Controlled-Release Coatings: EC-10/Peg 400

IR beads (925 g) from 3.A were coated with a solution of EC-10 (207.6 g) and PEG 400 (23.1 g) at a ratio of 90/10 dissolved in 85/15 acetone/water (9% solids) for a weight gain of 20%, following the procedures disclosed in Example 2.B above. The coated beads were then further coated with a seal coat of Klucel LF (applied as a solution in 85/15 acetone/water) for a 2% weight gain to produce SR beads (Lot# 1272-074).

Additional SR beads (lot# 1272-121) were prepared by coating IR beads (925 g) from 3.A, above with a solution of EC-10 (424.6 g) and PEG 400 (34.4 g) at a ratio of 92.5/7.5 dissolved in 85/15 acetone/water for a weight gain of 32.5% and further applying a sealant coating (2% w/w) of Klucel LF, following the procedures disclosed in Example 2.B above. Samples were pulled at intermediate coating levels (e.g., 30%) to determine drug release profiles at different coating levels.

### 3.C Melperone HCl TPR Beads (Controlled-Release Coating: 20% Weight Gain)

IR beads (1000 g) from 3.A, above, were coated with a lag-time coating of EC-10/HP-55/TEC at a ratio of 60/25/15 for a weight gain of 20%, followed by a seal coat of Klucel LF (applied as a solution in 85/15 acetone/water) for at 2% weight gain, as disclosed in Example 2.D, above, to produce TPR beads (Lot# 1272-111).

### 3.D Melperone HCl IR Beads (Drug Load: 25% by Weight)

20-25 mesh sugar spheres (1250 g) were layered with melperone hydrochloride (428.1 g) from a 50/50 acetone/water solution (1427 g each, 15% solids) in a Glatt GPCG 3 and further coated with a protective seal coat of hydroxypropylcellulose (Klucel LF dissolved in 85/15 acetone/water at 7% solids) for a 2% weight gain to form IR beads following the procedures disclosed in Example 2.A above.

### 3.E Melperone HCl SR Beads

IR beads (1200 g) from 3.D above for were coated with a solution of 633.4 g EC-10 and 33.3 g PEG 400 at a ratio of 95/5 dissolved in 85/15 acetone/water (10% solids) for a weight gain of 35%. The resulting SR beads were provided with a seal coat of hydroxypropylcellulose (Klucel LF, dissolved in 85/15 acetone/water at 7% solids) for a 2% weight gain to produce SR beads (Lot# 1272-109).

FIG. 5 shows the drug release profiles from various SR beads (Lot# 1272-074 at a coating of 20% w/w on 25-30 mesh sugar spheres, Lot# 1272-121 at a coating of 30% w/w on 25-30 mesh sugar spheres, and Lot# 1272-109 at a coating of 35% w/w on 20-25 mesh sugar spheres) and TPR beads (Lot# 1272-111 at a coating of 20% w/w on 25-30 mesh sugar spheres). The ratios of EC-10/PEG 400 or EC-10/HP-55/TEC and coating levels are given in parentheses in the legend of FIG. 5.

### Example 4

### 4.A Melperone HCl IR Beads (Drug Load: 25% by Weight)

Melperone HCl (1027.4 g) was slowly added to 50/50 acetone/water (3425 g each) while stirring to dissolve. A Glatt GPCG 5 equipped with a 9″ bottom spray Wurster 10″ column, 25 mm partition gap, 16 mm tubing, bottom air distribution ‘D’ plate with 200 mesh screen, atomization air pressure of 1.3 bar and 1.0 mm nozzle diameter was charged with 3000 g of 25-30 mesh sugar spheres, which were then sprayed with the melperone coating solution at an initial rate of 10 mL/min with a stepwise increase to 36 mL/min while maintaining the target fluidization air volume at 100 CFM and product temperature at 33° C. A sealant solution of hydroxypropylcellulose (Klucel LF, dissolved in 85/15 acetone/water, 7% solids) was applied to give a 2% weight gain, and the IR beads were dried to remove residual moisture and acetone.

### 4.B Melperone Hydrochloride SR Beads (Drug Load: 20% by Weight)

Melperone hydrochloride IR beads (1000 g) from 4.A were coated with a solution of EC-10 and PEG 400 at a ratio of 90/10 dissolved in 85/15 acetone/water (10% solids) for a weight gain of 25% following the procedures as disclosed in 3.B, above, and dried in the Glatt GPCG 5 at approximately 33° C. for 5 minutes to drive off residual solvent. The resulting SR beads were designated Lot# 1272-173. IR beads coated with 90/10 EC-10/PEG 400 at 20% by weight, using similar methods were designated Lot# 1272-175, and IR beads coated with 92.5/7.5 EC-10/PEG 400 at 25% by weight were designated Lot# 1295-024. The dried beads were sieved to discard any doubles, if formed.

### 4.C Melperone Hydrochloride CR Capsules

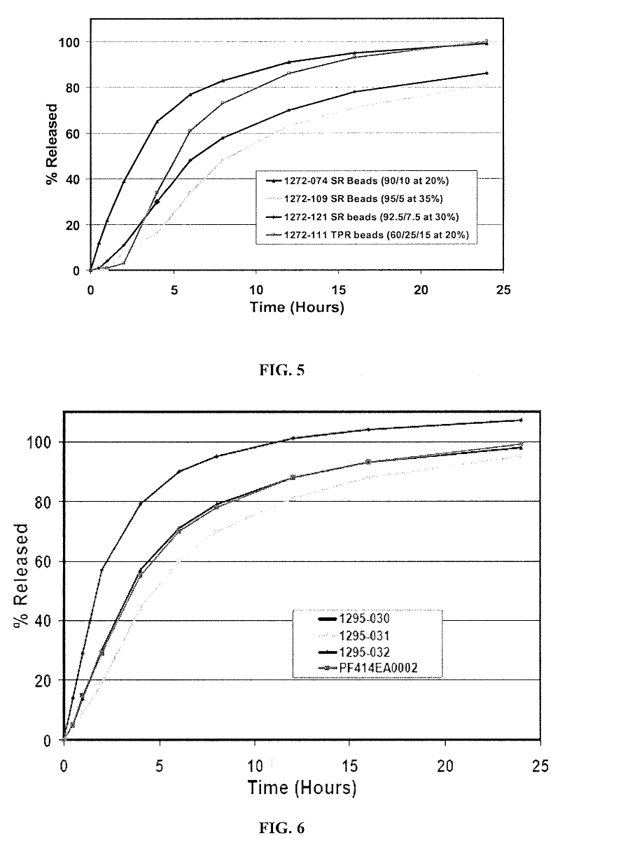

Appropriate amounts of the SR beads of 4.B were filled into hard-gelatin capsules using MG-2 Futura 8400 equipped with a 4.8 mm dosing ring to produce about 6000 capsules containing 50 mg melperone hydrochloride. Approximately 267.4 mg of SR beads Lot# 1272-173 were filled into a hard-gelatin capsule to produce CR Capsule Lot# 1295-030. Approximately 247.5 mg of SR beads Lot# 1272-175 were filled into a hard-gelatin capsule to produce CR Capsule Lot# 1295-031. Approximately 287.4 mg of SR beads Lot# 1295-024 were filled into a hard-gelatin capsule to produce CR Capsule Lot# 1295-032. FIG. 6 demonstrates the in vitro drug release profiles from these capsules, matching the deconvoluted release profiles calculated from the two-compartment model (shown in FIG. 3).

### Example 5

### 5.A Melperone HCl IR Beads (Drug Load: 20% W/W)

Melperone hydrochloride (obtained from Lundbeck; 256.4 g) was slowly added to a 50/50 mixture of acetone and water (each 854.7 g) until dissolved, with stirring. A Glatt GPCG 3 equipped with a 6″ bottom spray Wurster 8″ column, ‘C’ bottom air distribution plate covered with a 200 mesh product retention screen, 1.0 mm port nozzle, was charged with 1000 g of 45-60 mesh sugar spheres, which were then sprayed with a melperone hydrochloride solution at an initial rate of 10 mL/min with a stepwise increase to 20 mL/min, at the inlet air volume set at 15 CFM, air atomization pressure of 1.00 bar while maintaining the product temperature of 32±2° C. Following rinsing with 50 g of acetone/water, the sealant solution of Klucel LF dissolved in 85/15 acetone/water (7% solids) was sprayed at 10 mL/min, and the beads were dried in the unit for 5 min to drive off residual solvents (including moisture). The IR beads were sieved to discard oversized (>500 μm or 35 mesh) beads and fines (<80 mesh).

### 5.B Melperone Hydrochloride SR Beads

Ethylcellulose (EC-10; 408.1 g) was slowly added to 3750 g of acetone while stirring until dissolved. Purified water (661.8 g) was added to the ethylcellulose solution, while stirring. Then dibutyl sebacate (33.1 g) was slowly added to the ethylcellulose solution and stirred for 10 min. The melperone IR beads (1000 g) from 5.A above, were transferred to Glatt GPCG 3 equipped with a 6″ bottom spray Wurster 8″ column, 1.0 mm nozzle port, and a bottom ‘C’ distribution plate covered with a 200 mesh product retention screen, and coated with the EC-10/DBS solution at a fluidization air volume of 15 CFM, atomization air pressure of 1.25 bar, target product temperature of 33° C., and a spray rate of about 10 mL/min with a stepwise increase to 20 mL/min. Samples were pulled at coating levels of about 15%, 20%, and 25% by weight for dissolution testing. Following an acetone rinse, a compressible coating of hydroxypropylcellulose (Klucel LF, dissolved in 85/15 acetone/water, 7% solids) was applied at a spray rate of 10 mL/min for a weight gain of about 2% to yield SR beads.

### 5.C Rapidly Dispersing Microgranules

The rapidly dispersing microgranules comprising a sugar alcohol (e.g., mannitol) and a disintegrant (e.g., crospovidone) at a ratio of from about 90/10 to about 99/1 were prepared following the procedure disclosed in the co-pending US Patent Application Publication No. U.S. 2005/0232988, published Oct. 20, 2005, the contents of which are hereby incorporated by reference in its entirety. For example, D-mannitol (152 kg) with an average particle size of approximately 20 μm or less (Pearlitol 25 from Roquette, France) was blended with 8 kg of cross-linked povidone (Crospovidone XL-10 from ISP) in a high shear granulator (GMX 600 from Vector) and granulated with purified water (approximately 32 kg) and wet-milled using a Comil from Quadro, and tray-dried for an LOD (loss on drying) of less than about 0.8%. The dried granules were sieved and oversize material was milled to produce rapidly-dispersible microgranules with an average particle size in the range of approximately 175-300

### 5.D Melperone Hydrochloride ODT CR

The rapidly dispersing microgranules of 5.0 (1235.1 g) were blended with the Melperone HCl SR beads (856.2 g) of 5.B, above, and other pre-blended pharmaceutically acceptable ingredients (i.e., peppermint flavor (25.0 g), sucralose (8.8 g), crospovidone (125.0 g), and microcrystalline cellulose (250.0 g Avicel PH101) at a ratio of rapidly dispersing microgranules to SR beads of about 2:3 in a twin shell V-blender for a sufficient time to obtain a homogeneous compression blend. The blend was compressed using a production-scale tablet press equipped with an external lubrication system to produce ODTs (Lot# 1295-055) containing 50 mg melperone HCl as SR beads, with a mean tablet weight of 1008 mg, a mean hardness of about 46 N, and a friability of about 0.27%. Melperone HCl ODT CR, 50 mg thus produced rapidly disintegrated in the oral cavity, creating a smooth, easy-to-swallow suspension comprising coated melperone HCl beads and providing a target profile suitable for a once-daily dosing regimen. Another prototype batch of ODT CR (Lot# 1295-161) was prepared following the procedures disclosed in 5.A to 5.D, above.

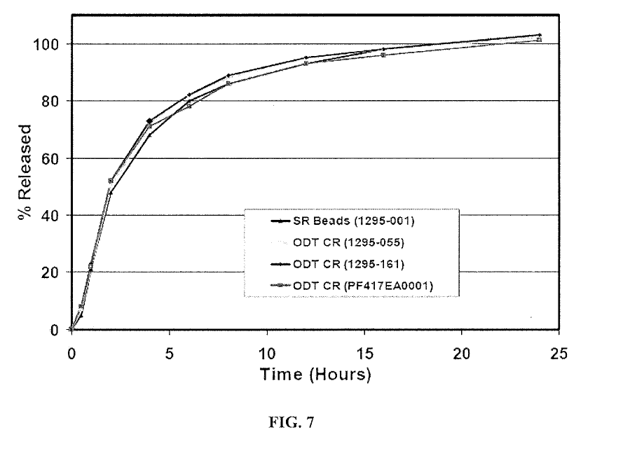

FIG. 7 shows the drug release profiles of Melperone HCl SR beads (Lot# 1295-001, prepared as described in Examples 5.A and 5.B, above) and ODT CR prototypes (Lot# 1295-055 and Lot# 1295-161) comprising SR beads from duplicate lots. The SR beads (Lot# 1295-001) and the corresponding ODT CR (Lot# 1295-055) appear to exhibit similar drug release profiles, indicating minimal membrane fracture (if any), during compression to form ODT tablets.

### Example 6

### 6.A Melperone HCl CR (Capsule), PF414EA0002

Melperone HCl CR Capsules (PF414EA0002) containing 50-mg of melperone hydrochloride as SR beads were prepared by coating IR beads at a drug load of 25 w/w with ethylcellulose and polyethylene glycol (PEG 400) at a ratio of 92.5/7.5 for a weight gain of 25% w/w, as described in Example 4 (see description of CR capsule Lot #1295-032 and SR beads, Lot # 1295-024, above). Drug release profiles for these capsules (PF414EA0002) (2-stage dissolution tests) are shown in FIG. 6.

### 6.B Melperone HCl ODT CR, PF417EA0001

Melperone Hydrochloride ODT CR(PF417EA0001) containing 50-mg melperone hydrochloride as SR beads prepared by coating IR beads at a drug load of 20% w/w with ethylcellulose and dibutyl sebacate (DBS) at a ratio of 92.5/7.5 for a weight gain of 30% w/w as disclosed in Example 5. The drug release profile for this ODT (2-stage dissolution test) is shown in FIG. 7.

From the above it is apparent that it is possible to prepare multiparticulate pharmaceutical drug delivery systems, such as CR Capsules or ODT CR dosage forms comprising weakly basic melperone hydrochloride, that is very soluble under acidic to neutral pH conditions, and that exhibit plasma profiles suitable for a once-daily dosing regimen, thereby helping improve patient compliance. The orally disintegrating tablets incorporating the SR beads rapidly disintegrate into a smooth, easy-to-swallow suspension on contact with the saliva in the oral cavity of patients, have a non-gritty mouthfeel and no aftertaste, and provide target plasma concentration-time profiles upon oral administration with clinically significant efficacy and which promote improved patient compliance.

We claim:

1. A pharmaceutical composition comprising one or more populations of controlled-release particles, wherein at least one population of controlled-release particles comprises:
(a) a core comprising melperone or a pharmaceutically acceptable salt, solvate, and/or ester thereof; and
(b) a controlled-release coating disposed over said core, wherein the controlled-release coating comprises a water-insoluble polymer.

2. The pharmaceutical composition of claim 1, wherein melperone is present as melperone hydrochloride.

3. The pharmaceutical composition of claim 1, further comprising a compressible coating disposed on said controlled-release coating.

4. The pharmaceutical composition of claim 3, wherein said compressible coating comprises at least one hydrophilic polymer.

5. The pharmaceutical composition of claim 4, wherein said at least one hydrophilic polymer is selected from the group consisting of hydroxypropylcellulose, poly(vinyl acetate-vinyl pyrrolidone), polyvinyl acetate, ethylcellulose, and mixtures thereof.

6. The pharmaceutical composition of claim 3, wherein said compressible coating comprises up to about 10% of said controlled-release particle.

7. The pharmaceutical composition of claim 1, wherein said water-insoluble polymer is selected from the group consisting of ethylcellulose, cellulose acetate, cellulose acetate butyrate, polyvinyl acetate, neutral methacrylic acid-methylmethacrylate copolymers, and mixtures thereof.

8. The pharmaceutical composition of claim 1, wherein the water-insoluble polymer comprises ethylcellulose.

9. The pharmaceutical composition of claim 1, where said controlled-release coating further comprises a plasticizer.

10. The pharmaceutical composition of claim 1, wherein the controlled-release layer further comprises a water-soluble polymer.

11. The pharmaceutical composition of claim 10, wherein the weight of the controlled-release layer ranges from about 15% to about 40% of the total weight of said controlled-release particle.

12. The pharmaceutical composition of claim 10, wherein said water-soluble polymer is selected from the group consisting of povidone, polyethylene glycol, hydroxypropyl methylcellulose, and hydroxypropylcellulose.

13. The pharmaceutical composition of claim 10, wherein the ratio of said water-insoluble polymer to said water-soluble polymer ranges from about 95:5 to about 50:50 based on the weight of said controlled-release particle.

14. The pharmaceutical composition of claim 10, wherein said water-insoluble polymer comprises ethylcellulose and said water-soluble polymer comprises polyethylene glycol.

15. The pharmaceutical composition of claim 1, wherein the controlled-release coating further comprises an enteric polymer.

16. The pharmaceutical composition of claim 15, wherein the ratio of said water-insoluble polymer to said enteric polymer ranges from about 9:1 to about 1:3.

17. The pharmaceutical composition of claim 1, further comprising an outer, lag-time coating disposed over said controlled-release coating.

18. The pharmaceutical composition of claim 1, wherein the core comprises an inert bead coated with melperone or a pharmaceutically acceptable salt, ester, and/or solvate thereof.

19. The pharmaceutical composition of claim 18, wherein the melperone coating ranges from about 20% to about 30% by weight of the coated inert bead.

20. The pharmaceutical composition of claim 18, wherein said inert bead comprises sugar, microcrystalline cellulose, lactose-microcrystalline cellulose, mannitol-microcrystalline cellulose, or silicon dioxide.

21. The pharmaceutical composition of claim 18, wherein said melperone coating further comprises a polymeric binder.

22. The pharmaceutical composition of claim 21, wherein said polymeric binder is selected from the group consisting of hydroxypropylcellulose, povidone, methylcellulose, hydroxypropyl methylcellulose, carboxyalkylcellulose, polyethylene oxide, starch, and a polysaccharide.

23. The pharmaceutical composition of claim 1, wherein said pharmaceutical composition when dissolution tested using United States Pharmacopoeia Apparatus 2 (paddles @ 50 rpm) in a 2-stage dissolution media (700 mL of 0.1N HCl for the first 2 hrs followed by testing in 900 mL buffer at pH 6.8 obtained by adding 200 mL of a pH modifier) at 37° C. exhibits a drug release profile substantially corresponding to the following pattern:
after an hour, no more than about 30% of the total melperone is released;
after 4 hrs, from about 30-70% of the total melperone is released;
after 8 hrs, from about 50-90% of the total melperone is released; and
after 12 hrs, not less than 80% of the total melperone is released.

24. A capsule comprising the composition of claim 1.

25. A method of preparing the pharmaceutical composition of claim 1, comprising:
(a) preparing a core comprising melperone or a pharmaceutically acceptable salt, ester, and/or solvate thereof; and
(b) coating the core with a controlled-release coating comprising a water-insoluble polymer.

26. The method of claim 25, wherein said step (a) comprises layering an inert bead with a solution comprising melperone and/or a pharmaceutically acceptable salt, ester, or solvate thereof, and optionally a polymeric binder.

27. The method of claim 25, wherein the coating of said step (b) further comprises a water-soluble polymer or an enteric polymer.

28. The method of claim 25, further comprising:
(c) applying a compressible coating comprising at least one hydrophilic polymer, wherein said compressible coating in disposed over said controlled-release coating.

29. The method of claim 25, further comprising coating said controlled-release particles with a second controlled-release layer comprising a water-insoluble polymer and/or an enteric polymer.

30. A method of treating psychosis and/or schizophrenia comprising administering to a patient in need thereof the composition of claim 1.
